(19) United States
(12) Patent Application Publication
(10) Pub. No.: US 2023/0108417 A1
(43) Pub. Date: Apr. 6, 2023

(54) HEALTH SUPPORT SYSTEM, HEALTH SUPPORT METHOD, AND STORAGE MEDIUM

(71) Applicant: NEC Corporation, Tokyo (JP)

(72) Inventor: Shigeki YAMASHITA, Tokyo (JP)

(73) Assignee: NEC Corporation, Tokyo (JP)

(21) Appl. No.: 17/903,686

(22) Filed: Sep. 6, 2022

(30) Foreign Application Priority Data
Sep. 29, 2021 (JP) 2021-158567

(51) Int. Cl. G09B 19/00

(52) U.S. Cl. CPC G09B 19/00

(57) ABSTRACT

A health support system according to an aspect of the present disclosure includes: at least one memory configured to store instructions; and at least one processor configured to execute the instructions to: receive an activity record representing behavior of a user of a health application downloaded using a utilization code for downloading the health application, the utilization code being attached to information on a result of medical examination; detect a transformation of the behavior of the user from the received activity record; generate behavior transformation information representing the transformation; and output the generated behavior transformation information.

This application is based upon and claims the benefit of priority from Japanese Patent Application No. 2021-158567, filed on Sep. 29, 2021, the disclosure of which is incorporated herein in its entirety by reference.

### TECHNICAL FIELD

The present disclosure relates to a health support technique.

### BACKGROUND ART

To maintain sustainability of social security, extension of healthy life is effective. In order to extend the healthy life, it is necessary to grasp a health state and transform the behavior according to the health state.

Japanese Patent Application Laid-open Publication No. 2002-063278 discloses a health evaluation system that evaluates a transition of a health level of a listed company from a result of comparison between a transition of medical examination data of an evaluated person, a health standard model based on a transition of medical examination data of a healthy person, and a disease model based on a transition of medical examination data of a non-healthy person.

Japanese Patent Application Laid-open Publication No. 2010-113668 discloses a health guidance support device that extracts guidance items based on a result of medical examination of a medical examinee, calculates a guidance effect of the extracted guidance items based on past results, and selects guidance items based on the extracted guidance items and the calculated guidance effect.

Japanese Patent Application Laid-open Publication No. 2014 -119817 discloses a health management prediction system that simulates a health index and a health risk of an individual based on test value data and lifestyle data of the individual and predetermined parameters of the test value data and the lifestyle data.

### SUMMARY

One object of the present disclosure provides a health support system and the like capable of grasping information on a behavior transformation regarding health of a subject.

A health support system according to an aspect of the present disclosure includes: at least one memory configured to store instructions; and at least one processor configured to execute the instructions to: receive an activity record representing behavior of a user of a health application downloaded using a utilization code for downloading the health application, the utilization code being attached to information on a result of medical examination; detect a transformation of the behavior of the user from the received activity record; generate behavior transformation information representing the transformation; and output the generated behavior transformation information.

A health support method according to an aspect of the present disclosure includes: receiving an activity record representing behavior of a user of a health application downloaded using a utilization code for downloading the health application, the utilization code being attached to information on a result of medical examination; detecting a transformation of the behavior of the user from the received activity record; generating behavior transformation information representing the transformation; and outputting the generated behavior transformation information.

A non-transitory computer-readable storage medium according to an aspect of the present disclosure stores a program causing a computer to execute: record receiving processing of receiving an activity record representing behavior of a user of a health application downloaded using a utilization code for downloading the health application, the utilization code being attached to information on a result of medical examination; generation processing of detecting a transformation of the behavior of the user from the received activity record, and generating behavior transformation information representing the transformation; and record output processing of outputting the generated behavior transformation information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a health support system according to the first example embodiment of the present disclosure;

FIG. 2 is a flowchart illustrating an example of an operation of the health support system according to the first example embodiment of the present disclosure;

FIG. 3 is a block diagram illustrating an example of a configuration of a health support system according to a second example embodiment of the present disclosure;

FIG. 4 is a block diagram illustrating a second example of a configuration of the health support system according to the second example embodiment of the present disclosure;

FIG. 5 is a block diagram illustrating a third example of a configuration of the health support system according to the second example embodiment of the present disclosure;

FIG. 6 is a block diagram illustrating a fourth example of the configuration of the health support system according to the second example embodiment of the present disclosure;

FIG. 7 is a flowchart illustrating an example of an operation of generating a code of a health support system according to a second example embodiment of the present disclosure;

FIG. 8 is a flowchart illustrating an example of an operation of performing a health simulation of the health support system according to the second example embodiment of the present disclosure;

FIG. 9 is a flowchart illustrating an example of an operation of generating behavior transformation information of the health support system according to the second example embodiment of the present disclosure;

FIG. 10 is a block diagram illustrating an example of a configuration of a health support system according to a first modified example of the second example embodiment of the present disclosure; and

FIG. 11 is a diagram illustrating an example of a hardware configuration of a computer capable of achieving each of the health support systems according to the example embodiment of the present disclosure.

### EXAMPLE EMBODIMENT

Next, example embodiments of the present disclosure will be described in detail with reference to the drawings.

### First Example Embodiment

First, a first example embodiment of the present disclosure will be described in detail with reference to the drawings.

### Configuration

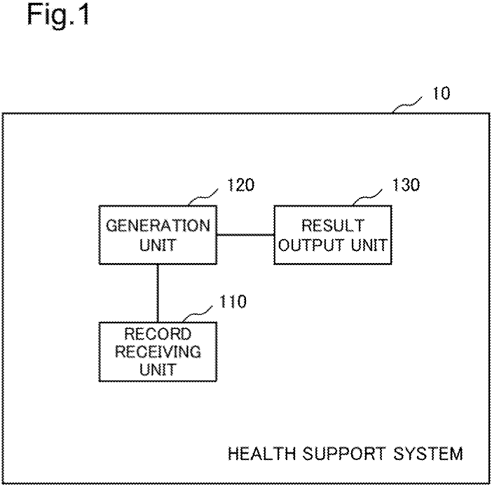

FIG. 1 is a block diagram illustrating an example of a configuration of a health support system according to a first example embodiment of the present disclosure. In the example illustrated in FIG. 1, a health support system 10 includes a record receiving unit 110, a generation unit 120, and a result output unit 130. The record receiving unit 110 receives an activity record of a user of a health application downloaded using a utilization code for downloading the health application, which is attached to information on a result of medical examination. The generation unit 120 generates behavior transformation information of a user based on the received activity record. The result output unit 130 outputs the generated behavior transformation information.

The health application is downloaded to a user’s terminal device using, for example, a utilization code such as a quick response (QR) code (registered trademark) attached to a document for notifying the result of the medical examination, and is installed on the terminal device. The utilization code represents a source (for example, unified resource identifier (URI) of an installation program of a health application) of the health application. The utilization code is read by an imaging device mounted on the terminal device and decoded by a reader application under control of the reader application operating on the terminal device. The health application is downloaded, by a user, from the URI obtained by, for example, decoding to the terminal device. The downloaded health application is installed on the terminal device by the user. In the following description, the operation of the terminal device operating by controlling the health application may be described as the operation of the health application.

The health application is an application that records information on a user’s behavior and transmits the recorded information on the user’s behavior to a management device. The health application operates, for example, in a terminal device such as a smartphone held by a user.

The information on the user’s behavior includes, for example, information on a time zone of meal and snack, information on a time zone of exercise, information on a time zone of sleep, and the like. The information on the user’s behavior may include, for example, calories, the amount of each nutrient, the number of times, an interval, and the like as information on the contents of the user’s meal and snack. The information on the user’s behavior may include, for example, a magnitude of a load, duration, the number of times, a type, a moving distance, and the like as the information on the contents of the user’s exercise.

The management device collects the information on the user’s behavior from the terminal device on which the health application of the user operates, and collects the collected information (that is, the above-described activity record) on the user’s behavior to the record receiving unit 110 of in the health support system 10.

The record receiving unit 110 receives an activity record representing the user’s behavior, and transmits the received activity record to the generation unit 120.

The generation unit 120 receives the activity record representing the user’s behavior from the record receiving unit 110. As described above, the generation unit 120 generates behavior transformation information representing the transformation of the user’s behavior based on the received activity record. The behavior transformation information of the user is information on the transformation of the user’s behavior. In other words, the generation unit 120 detects the transformation of the user from the received activity record representing the user’s behavior. The generation unit 120 generates the behavior transformation information representing the detected transformation of the user. The behavior transformation information will be described in detail later. The generation unit 120 transmits the generated behavior transformation information to the result output unit 130.

The result output unit 130 receives the behavior transformation information of the user from the generation unit 120, and outputs the received behavior transformation information.

An output destination of the behavior transformation information may be, for example, a local government (specifically, an information processing device managed by a local government) in which a user resides. In this case, the behavior transformation information may be used by the local government to draft measures such as events and health missions to improve health conditions of residents. In this case, the event is, for example, an event for maintaining or improving the health conditions. The health mission is, for example, a behavioral object to maintain or improve the health conditions. The event may be an event that aims to achieve one or more health missions. The behavior transformation information may be notified to a user or a resident including the user by the local government.

The output destination of the behavior transformation information may be, for example, a provider (specifically, a management device managed by a provider) that provides a health application. In this case, the behavior transformation information may be notified to the user by the provider (specifically, for example, a management device) of the health application, for example, via the health application. The behavior transformation information may be used by the provider of the health application to draft measures such as events and health missions to improve the health state of the user of the health application. The measures such as these events and health missions may be provided to a user, for example by the health application.

The output destination of the behavior transformation information is not limited to these examples.

### Operation

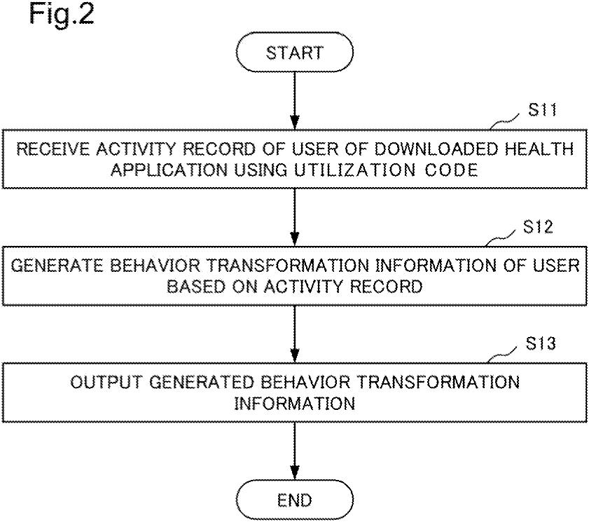

FIG. 2 is a flowchart illustrating an example of an operation of the health support system according to the first example embodiment of the present disclosure. In the example illustrated in FIG. 2, first, the record receiving unit 110 receives the activity record of the user of the health application downloaded using the utilization code (step S11). Next, the generation unit 120 generates the behavior transformation information of the user based on the activity record (step S12). The result output unit 130 outputs the generated behavior transformation information (step S13).

### Effect

The present example embodiment has the effect of grasping information on a transformation of subject’s behavior related to health. The reason is that the generation unit 120 generates the behavior transformation information of the user based on the activity record of the user.

### Details of Utilization Code

The utilization code may be generated to further represent a user ID. When downloading the health application, the terminal device may transmit the user ID to the management device together with a request to download the health application. The request to download the health application may include the user ID.

### Specific Example of Behavior Transformation Information

The behavior transformation information may be statistical information on behavior transformations of a plurality of users. The behavior transformation information may be information (also referred to as individual information in the following description) on behavior transformations of each user. Hereinafter, specific examples of these statistical information and individual information will be described.

### Example of Statistical Information

The statistical information on the transformation of the user’s behavior may be the latest number of users whose behavior has been transformed. The latest number may be the latest cumulative number. The latest number may be the latest increase number (that is, the number increased after timing when the previous increase number is calculated). The statistical information on the transformation of the user’s behavior may be the latest number of users whose behavior has been transformed.

In these cases, the management device of the health application transmits the activity record of the user obtained from the terminal device that executes the health application to the record receiving unit 110. The record receiving unit 110 receives the activity record of the user from the management device. The generation unit 120 identifies the user whose behavior has been transformed in the activity record of the user. The generation unit 120 generates the statistical information on the transformation of the user’s behavior from the specified user information, for example, at a predetermined timing. Specifically, the generation unit 120 may generate the statistical information on the transformation of the user’s behavior at predetermined time intervals. When the record receiving unit 110 receives the activity record of the user from the management device, the generation unit 120 may generate the statistical information on the transformation of the user’s behavior. When the result output unit 130 generates the behavior transformation information of the user, the result output unit 130 may output the generated behavior transformation information of the user.

The statistical information on the transformation of the user’s behavior may be a ratio of the latest number of users whose behavior has been transformed to the number of users for whose utilization codes have been generated. The statistical information on the transformation of the user’s behavior may be a transition of the ratio of the latest number of users whose behavior has been transformed to the number of users whose utilization codes have been generated.

In this case, the record receiving unit 110 further receives, for example, from the device that generates the utilization code, the information (for example, a list of user IDs, the number of users whose utilization codes have been generated, or the like) on the user whose utilization codes have been generated. The generation unit 120 specifies the number of users whose utilization codes have been generated from the information on the user whose utilization codes have been generated. The generation unit 120 identifies the number of users whose behavior has been transformed in the activity record of the user. The generation unit 120 generates, from the number of users whose utilization codes have been generated and the number of users whose behavior has been transformed, the statistical information on the transformation of the user’s behavior at the above-described predetermined timing.

### Example of Behavior Transformation

The transformation of behavior, that is, the behavior transformation may be the use of the health application downloaded using the utilization code. The use of the health application may be the download of the health application using the utilization code. The use of the health application may activate the health application downloaded using the utilization code at least once and register to receive services by the health application. The use of the health application may be the continuous use of the health application downloaded using the utilization code.

When the behavior transformation is the use of the health application downloaded using the utilization code and the use of the health application is the download of the health application using the utilization code, the record receiving unit 110 operates as follows, for example.

The record receiving unit 110 receives, as the activity record, a list (specifically, a list of user identifiers (hereinafter, also referred to as user IDs) assigned to a user of users who have used the health application. In this case, the user who uses the health application is a list of users who download the health application. The management device of the health application transmits a list of received user IDs together with the request for downloading the health application to the record receiving unit 110 as the information on the behavior transformation. When the management device receives the same user ID twice or more together with a request to download the health application, the management device generates a list of user IDs in such a way that the same user ID is not included in the list twice or more.

When the behavior transformation starts using the health application downloaded using the utilization code, and the start of the use of the health application is the first activation of the downloaded health application, the management device operates as follows.

The management device of the health application detects the start of the use of the health application downloaded using the utilization code, and specifies the user ID of the user who downloads the health application for which the start of the use is detected. In this case, for example, the terminal device that executes the health application may be configured to hold the user ID and transmit the user ID to the management device at the time of the first activation of the health application. The management device may receive the user ID from the terminal device at the first activation of the health application. The management device may specify the user ID received from the terminal device at the time of the first activation of the health application as the user ID of the user who downloads the health application for which the start of the use is detected. The management device transmits a list of user IDs of the specified users to the record receiving unit 110.

The record receiving unit 110 receives, as the activity record, a list of user IDs of the specified users, that is, a list of users who use the health application. In this case, the user who has used the health application is a list of users who first activates the downloaded health application.

The behavior transformation may be the continuous use of the health application downloaded using the utilization code. The continuous use may activate, for example, the health application at least once within a predetermined time in the past. The continuous use may activate, for example, the health application twice or more and activate the health application at least once within a predetermined time in the past. In this case, the behavior transformation information is the statistical information on the number of users who continuously use the health application downloaded using the utilization code.

In this case, the management device of the health application transmits, as the above-described activity record, the history of the activation of the health application to the record receiving unit 110. The history of the activation of the health application is, for example, a list of combinations of a user ID of a user who activates the health application and information representing the date and time when the health application is activated. In this case, the terminal device on which the health application is installed may hold the user ID and may transmit the user ID to the management device each time the health application is activated. The management device holds, as the history of the activation of the health application, a list of combinations of the user ID received from the terminal device on which the health application is activated and the time when the user ID is received. In this case, the management device considers that the time when the user ID is received is the time when the health application is activated in the terminal device holding the user ID.

The record receiving unit 110 receives, as the above-described activity record, the history of the activation of the health application from the management device. The generation unit 120 specifies the number of users who continuously use the health application from the history of the activation of the health application. The generation unit 120 generates the statistical information on the number of users who continue to use the specified health application, and the number of users who continue to use the health application downloaded using the utilization code.

The behavior transformation may be that a predetermined type of behavior in a predetermined state for the type of behavior. Specifically, a predetermined type of behavior may be a combination of at least one of a time zone of food intake, the number of times of food intake, an interval of food intake, the amount of food intake, calories of food intake, and an amount of each nutrient of food intake. The predetermined type of behavior may be a combination of at least any of magnitude of exercise load, duration of exercise, the number of times per given time of exercise, and the like. The predetermined type of behavior may be to bringing a predetermined type of physical condition into a state defined for the type of physical condition. In this case, the predetermined type of physical condition may be a combination of at least any of a body weight, a subcutaneous fat thickness, a bone mass, a bone density, and the like. The behavior transformation may be a behavior that can contribute to the improvement in the health condition. The behavior that can contribute to the improvement in the health condition may be, for example, going to bed at a time included in a time zone considered to be less likely to occur in the deterioration in the health condition. The behavior that can contribute to the improvement in the health condition may be, for example, waking up at a time included in a time zone considered to be less likely to occur in the deterioration in the health condition. The behavior that can contribute to the improvement in the health condition may be, for example, taking a sleep length considered to be less likely to occur in the deterioration in the health condition. The behavior that can contribute to improving health may be, for example, weighing at certain time zone. The behavior that can contribute to improving health may be, for example, taking three meals a day. The behavior that can contribute to improving the health condition may be, for example, that the time of taking three meals is included in predetermined time zone. The time of taking a meal may be any one of the time of starting a meal, the time of ending a meal, and the time determined by a predetermined method for a time zone from the start to the end of a meal. The behavior transformation may be a combination of examples of the behavior transformation described above.

In this case, the behavior transformation may specifically be that the combination representing a predetermined type of behavior transformation is from a state in which the condition defined for the combination is not satisfied to a state in which the condition is satisfied. The condition defined for the combination is, for example, that the conditional expression regarding values of elements included in the combination is satisfied.

In this case, the health application transmits the information on the above-described predetermined type of behavior to the management device together with, for example, a user ID. The management device stores the received information on the predetermined type of behavior as an activity record for each user ID.

The record receiving unit 110 acquires the activity record from the management device. The generation unit 120 specifies the user whose behavior has been transformed from the acquired activity record. The generation unit 120 generates the statistical information on the number of users whose behavior has been transformed.

The behavior transformation may be that a predetermined type of behavior is in a predetermined state for that type of action, and that state continues (in other words, behavior after transformation continues).

Even in this case, the record receiving unit 110 acquires the activity record from the management device. The generation unit 120 specifies the user whose behavior has been transformed from the acquired activity record. Further, the generation unit 120 generates the statistical information on the number of users whose behavior has been transformed.

The behavior transformation may be that the above-described event causes a predetermined type of behavior to become a predetermined state for that type of behavior.

In this case, the record receiving unit 110 acquires the activity record from the management device. The record receiving unit 110 further acquires information on a period of an event from a device managed by an organizer of the event or information input by an operator of the health support system 10. The generation unit 120 specifies the user whose behavior has been transformed during the period of the event from the acquired activity record. Further, the generation unit 120 generates the statistical information on the number of users whose behavior has been transformed during the period of the event as the statistical information on the number of users whose behavior has been transformed by the event.

The behavior transformation may be to achieve a health mission. In this case, the behavior transformation information may be the statistical information on the number of users who have achieved the health mission.

In this case, for example, the health application transmits information to the management device indicating whether the user has achieved the health mission for each health mission. The management device receives the information indicating whether the health mission has been achieved and the user ID from the terminal device on which the health application is executed. The management device stores the received information indicating whether the health mission has been achieved as the activity record of the user for each user ID.

The record receiving unit 110 acquires the activity record from the management device. The generation unit 120 specifies the user whose behavior has been transformed from the acquired activity record. Further, the generation unit 120 generates the statistical information on the number of users whose behavior has been transformed.

The behavior transformation may be the reaction of the user to the information (in other words, the information provided by the health application) provided by the health application. The reaction to the information provision may be, for example, marking such as “like” or inputting a comment. In this case, the behavior transformation information may be the statistical information on the number of users who have reacted to the information provided by the health application.

In this case, the health application transmits information on the reaction to the information provision to the management device. The management device receives the information on the reaction to the information provision and the user ID from the terminal device on which the health application is executed. The management device stores the information on the reaction to the received information provision as the activity record of the user for each user ID.

The record receiving unit 110 acquires the activity record from the management device. The generation unit 120 specifies the user whose behavior has been transformed from the acquired activity record. Further, the generation unit 120 generates the statistical information on the number of users whose behavior has been transformed.

The behavior transformation may be a combination of two or more of the examples described above.

### Individual Information

Even if the generation unit 120 generates, as the behavior transformation information, the information indicating whether a behavior transformation has occurred for each user, instead of the statistical information on the number of users in which any of the above-described behavior transformations have occurred.

### Second Example Embodiment

Next, a second example embodiment of the present disclosure will be described in detail with reference to the drawings.

### Configuration

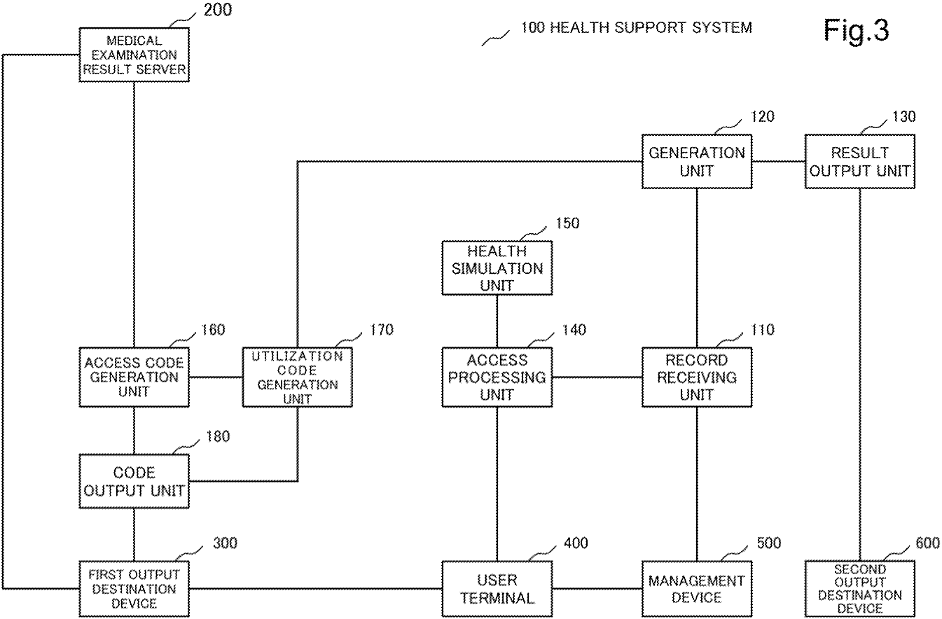

FIG. 3 is a block diagram illustrating an example of a configuration of a health support system according to a second example embodiment of the present disclosure. According to the example illustrated in FIG. 3, a health support system 100 includes a record receiving unit 110, a generation unit 120, a result output unit 130, an access processing unit 140, a health simulation unit 150, and an access code generation unit 160, a utilization code generation unit 170, and a code output unit 180. The health support system 100 is communicably connected to each of a medical examination result server 200, a first output destination device 300, a user terminal 400, a management device 500, and a second output destination device 600.

### Medical Examination Result Server 200

The medical examination result server 200 stores the result of the user’s medical examination. In the following description, the result of the medical examination may include the result of the medical interview (answer to the medical interview and result of examination by a doctor). The medical examination result server 200 may be, for example, a server managed by a local government. The medical examination result server 200 may store the result of the medical examination of the residents of the local government. The medical examination result server 200 may store personal information such as the user’s contact information (for example, address, e-mail address, and the like) and name, in addition to the result of the user’s medical examination. The result of the medical examination may include the results (specifically, a combination of the timing of the multiple medical examinations and the result of the medical examination) of a plurality of times of medical examinations.

The medical examination result server 200 outputs the result (specifically, the data of the result of the medical examination for each user) of the user’s medical examination to the access code generation unit 160 and the first output destination device 300 of the health support system 100. The medical examination result server 200 may supply the user’s personal information to the first output destination device 300.

### Access Code Generation Unit 160

The access code generation unit 160 receives the results of the medical examination for each user from the medical examination result server 200. The access code generation unit 160 generates an access code, which is a code representing a health simulation request to the health simulation unit 150, based on the results of the medical examination for each user. The health simulation request includes the result of the user’s medical examination used in the health simulation. The health simulation request may be represented by URI. The access code is, for example, a QR code. The access code generation unit 160 may generate an access code which is a code representing the result of the medical examination and the user ID. The access code generation unit 160 transmits the generated access code to the first output destination device 300.

The access code generation unit 160 generates a user ID for each user who receives the result of the medical examination, and assigns the generated user ID to the result of the user’s medical examination represented by the user ID. In this case, the access code generation unit 160 supplies the generated user ID to the medical examination result server 200, the utilization code generation unit 170, and the generation unit 120. As illustrated in FIG. 3, the access code generation unit 160 supplies the user ID to the generation unit 120 via the utilization code generation unit 170, but the access code generation unit 160 may directly supply the user ID to the generation unit 120. The medical examination result server 200 assigns an identifier (hereinafter referred to as a data ID) to the data of the results of the medical examination for each user, and confirms the result of the medical examination to which the data ID is assigned for each user to the access code generation unit 160. The access code generation unit 160 may associate the data ID with the user ID and supply data representing the association between the data ID and the user ID to the medical examination result server 200. In this case, the medical examination result server 200 receives data representing the association between the data ID and the user ID, and holds the received data representing the association between the data ID and the user ID. The user ID does not have to be assigned by the access code generation unit 160. An example in that case will be described later as a modified example.

### Utilization Code Generation Unit 170

The utilization code generation unit 170 generates the above-described utilization code for each user whose result of the medical examination is supplied to the access code generation unit 160. For example, the utilization code generation unit 170 receives the user ID from the access code generation unit 160, and generates the utilization code for each received user ID. As described above, the utilization code may include the information on the user ID.

The utilization code generation unit 170 transmits the generated utilization code to the code output unit 180.

### Code Output Unit 180

The code output unit 180 receives the access code of the user from the access code generation unit 160. The code output unit 180 receives the utilization code of the user from the utilization code generation unit 170. The code output unit 180 sends the access code of the user and the utilization code of the user to the first output destination device 300.

### First Output Destination Device 300

The first output destination device 300 receives the access code of the user and the utilization code of the user from the code output unit 180. The first output destination device 300 receives the results of the medical examination for each user from the medical examination result server 200. The first output destination device 300 generates a document (hereinafter, referred to as an information providing sheet) including an access code and a utilization code for each user, and outputs the generated information providing sheet. The information providing sheet may include the result of the medical examination. The first output destination device 300 is, for example, an information processing device managed by a local government.

The information providing sheet may be a sheet on which the access code, the utilization code, and the result of the medical examination are printed. In this case, the first output destination device 300 prints the information providing sheet. Then, for example, an employee of the local government may mail the information providing sheet to the user.

The information providing sheet may be electronic data in a predetermined format representing the access code, the utilization code, and the result of the medical examination. In this case, the first output destination device 300 may transmit the information providing sheet to the electronic mail address of the user as at least a part of the electronic mail. Specifically, the first output destination device 300 may transmit an e-mail to which a file of the information providing sheet is attached. The first output destination device 300 may transmit an e-mail in which the image of the access code and the image of the utilization code are incorporated in the text. In this case, the result of the medical examination may be incorporated in the text of the e-mail. The result of the medical examination may be attached to an e-mail as a file

### User Terminal 400

The user terminal 400 is an information processing device having a function of reading an access code and a utilization code and a function of interpreting the read access code and utilization code. The user terminal 400 is, for example, an information processing device that includes an imaging device and has an application of a QR code reader installed therein.

When the user causes the user terminal 400 to read the utilization code on the paper on which the access code and the utilization code are printed or on the screen on which the access code and the utilization code are displayed, the user terminal 400 reads the utilization code. The user terminal 400 interprets the read utilization code, and displays the screen for encouraging the download and installation of the health application as a result of interpreting the utilization code. When the user instructs the user terminal 400 to download and install the health application, the user terminal 400 downloads and installs the health application. When the user performs an operation of activating the health application on the user terminal 400, the user terminal 400 executes (in other words, activate) the health application. The health application may be installed from the management device 500. The health application may be installed from a server or the like (for example, a server of a vendor of an operating system (OS) of the user terminal 400, a server of a vendor of the user terminal 400, or the like) different from the management device 500.

When the user causes the user terminal 400 to read the access code on the paper on which the access code and the utilization code are printed or on the screen on which the access code and the utilization code are displayed, the user terminal 400 reads the access code. The user terminal 400 generates a health simulation request from the read access code. The user terminal 400 transmits the health simulation request to the health simulation unit 150 via the access processing unit 140. As described above, the health simulation request includes a result of medical examination used for the health simulation. Further, the user terminal 400 transmits the user ID to the access processing unit 140 in addition to the health simulation request. The user terminal 400 receives the result of the health simulation representing the result of simulating the future health condition from the health simulation unit 150 via the access processing unit 140. The user terminal 400 displays the received result of the health simulation.

The health application records the activity and state of the user. The health application may estimate the activity and state of the user and record the estimated activity and state by using a sensor such as an acceleration sensor mounted on the user terminal 400, a global positioning system (GPS) function for acquiring position information, and the like. The health application may record the activity and state input by the user using the input device of the user terminal 400. Data representing the recorded activity and state is referred to as the activity record. The activity record is the same as the activity record of the first example embodiment. The health application transmits the activity record to the management device 500. The activity record may be data called a so-called life log. The life log may be, for example, at least one of data input or generated by the user using the health application or data recorded by the health application without an active instruction by the user.

Further, the health application receives a notification of an event and a notification of a health mission from the management device 500. The health application receives information provision related to, for example, health or the like from the management device 500 (in other words, for example, information based on an article or the like is received). The health application displays the received notification and information on the display of the user terminal 400. The health application receives a reaction (for example, at least one of provision of a mark such as “like” to a provided article, a comment, and the like) of the user to the information provision from the user via the input device of the user terminal 400. The health application transmits the reaction of the user to the information provision to the management device 500. A plurality of types of health applications may acquire the user’s behavior and transmit the information representing the acquired user’s behavior to the management device 500. Each of the plurality of types of health applications may transmit the information representing the user’s behavior to the management device 500 defined according to the type of the health application among the plurality of management devices 500. Each of the plurality of management devices 500 may transmit the activity record representing the user’s behavior to the record receiving unit 110. A plurality of types of health applications may be provided by different providers. The plurality of management devices 500 may be managed by a plurality of different providers.

### Access Processing Unit 140

The access processing unit 140 receives the user ID and the health simulation request from the user terminal 400. As described above, the health simulation request may include the result of the medical examination. The health simulation request may include the result of the medical interview. The access processing unit 140 transmits the received user ID to the record receiving unit 110 as the user ID of the user who has performed the health simulation.

The access processing unit 140 transmits the result (alternatively, the result of the medical examination and the result of the medical interview) of the medical examination received as a part of the health simulation request to the health simulation unit 150. The access processing unit 140 receives the result of the health simulation from the health simulation unit 150, and transmits the received result of the health simulation to the user terminal 400. The access processing unit 140 may receive explanatory video generated from the result of the medical interview from the health simulation unit 150 and transmit the received explanatory video to the user terminal 400.

### Health Simulation Unit 150

The health simulation unit 150 receives the result of the medical examination from the access processing unit 140. Upon receiving the result of the medical examination from the access processing unit 140, the health simulation unit 150 executes the health simulation for predicting the future health condition using the received result of the medical examination. The method of the health simulation may be one of various existing methods that use the result of the medical examination to predict future health conditions. The health simulation unit 150 transmits the result of the health simulation to the access processing unit 140. The health simulation unit 150 may further receive the result of the medical interview from the access processing unit 140. Upon receiving the result of the medical interview, the health simulation unit 150 may create the explanatory video according to the result of the medical interview, and transmit the created explanatory video to the access processing unit 140. For example, the health simulation unit 150 determines whether the received result of the medical interview satisfies one or more conditions. The one or more conditions may be associated with partial video representing the explanation for the result of the medical interview, which is performed on the user when the result of the medical interview satisfies the conditions. For example, the health simulation unit 150 may generate the explanatory video by combining the partial video associated with the satisfied conditions.

### Management Device 500

The management device 500 is the same as the management device of the first example embodiment. The management device 500 receives the activity record of the user from the health application (specifically, the user terminal 400 that executes the health application), and holds the received activity record of the user. Then, the management device 500 transmits the activity record of the user to the record receiving unit 110 of the health support system 100.

The management device 500 may provide an installation program of the health application. Specifically, upon receiving the health application request from the user terminal 400, the management device 500 may transmit the health application installation program to the user terminal 400 that has transmitted the request.

### Record Receiving Unit 110

The record receiving unit 110 receives the activity record of the user from the management device 500. The record receiving unit 110 transmits the received activity record of the user to the generation unit 120. The record receiving unit 110 further receives the user ID of the user who has performed the health simulation from the access processing unit 140. The record receiving unit 110 transmits the received user ID of the user who has performed the health simulation to the generation unit 120. The record receiving unit 110 has the same function as the function of the record receiving unit 110 of the first example embodiment, and operates similarly to the record receiving unit 110 of the first example embodiment.

### Generation Unit 120

The generation unit 120 has the same function as the generation unit 120 of the first example embodiment, and operates similarly to the generation unit 120 of the first example embodiment. The generation unit 120 of the present example embodiment receives the user ID of the user who has executed the health simulation from the record receiving unit 110. In addition, the generation unit 120 receives the user ID of the user whose access code and utilization code are generated from the access code generation unit 160 directly or via the utilization code generation unit 170. In the present example embodiment, both the access code and the utilization code are generated for each of the users. Therefore, the user whose access code and utilization code are generated is the same as the user whose utilization code is generated.

As described above, the generation unit 120 generates the behavior transformation information of the user based on the received activity record. The generation unit 120 may further use at least one of the user ID of the user whose utilization code has been generated, the user ID of the user on which the health simulation has been executed, and the user ID of the user who has executed the health application to generate the behavior transformation information of the user. The behavior transformation information of the present example embodiment will be described in detail later. The generation unit 120 transmits the generated behavior transformation information to the result output unit 130.

### Result Output Unit 130

The result output unit 130 receives the behavior transformation information from the generation unit 120 and transmits the received behavior transformation information to the second output destination device 600.

### Second Output Destination Device 600

The second output destination device 600 receives the behavior transformation information from the result output unit 130. The second output destination device 600 presents the received behavior transformation information to a manager of the second output destination device 600.

The second output destination device 600 may be managed by a local government that manages the medical examination result server 200. In this case, the manager of the second output destination device 600 may refer to the presented behavior transformation information to devise other measures or the like that urges the behavior transformation for the medical examinee who has received the event, the health mission, and the medical examination. The manager of the second output destination device 600 may refer to the presented behavior transformation information and give advice for encouraging the behavior transformation for each user. The second output destination device 600 may use the presented behavior transformation information to generate, for each user, a recommendation for behavior transformation by any of various existing methods for generating health advice. The advice for encouraging the behavior transformation may be transmitted to, for example, the contact address of the user stored in the medical examination result server 200. Specifically, the advice for encouraging the behavior transformation may be printed on paper and mailed to the address of the user. The advice prompting the behavior transformation may be transmitted as an electronic file to the user’s e-mail address by e-mail.

The second output destination device 600 may be managed by a vendor that provides a health application. In this case, the management device 500 may operate as the second output destination device 600. The second output destination device 600 may be communicably connected to the management device 500. The manager of the second output destination device 600 may refer to the presented behavior transformation information and give advice for encouraging the behavior transformation for each user. The second output destination device 600 may use the presented behavior transformation information to generate, for each user, a recommendation for behavior transformation by any of various existing methods for generating health advice. The second output destination device 600 may present the advice for encouraging the user to transform the behavior to the user via the health application. In this case, the health application of the user terminal 400 may acquire the advice for encouraging the behavior transformation directly from the second output destination device 600 or via the management device 500, and display the acquired advice for encouraging the behavior transformation on the display device of the user terminal 400. The management device 500 may transmit the advice for encouraging the behavior transformation to the e-mail address of the user. In this case, the management device 500 may be configured to collect the electronic mail address of the user via the health application or the installation program of the health application.

### Configuration Example

The health support system 100 described above may be achieved by one information processing device. The health support system 100 may be achieved by a plurality of information processing devices each including at least one component and communicably connected. In the example illustrated below, each of the components of the health support system is the same as the component assigned the same name and the same reference numeral in FIG. 3.

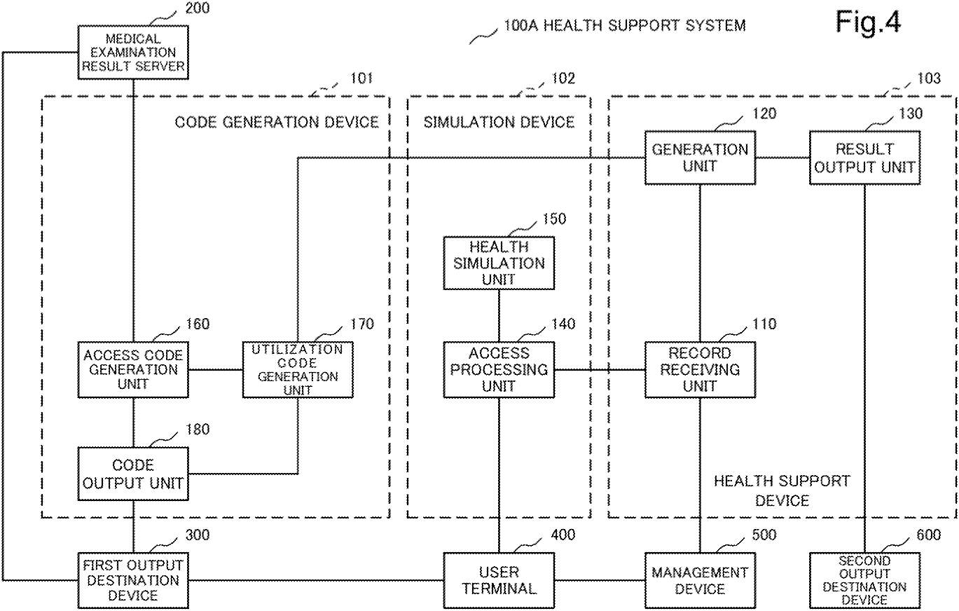

FIG. 4 is a block diagram illustrating a second example of a configuration of the health support system according to the second example embodiment of the present disclosure. In the example of FIG. 4, the health support system 100A includes a code generation device 101, a simulation device 102, and a health support device 103. The code generation device 101 includes an access code generation unit 160, a utilization code generation unit 170, and a code output unit 180. The simulation device 102 includes an access processing unit 140 and a health simulation unit 150. The health support device 103 includes the record receiving unit 110, the generation unit 120, and the result output unit 130.

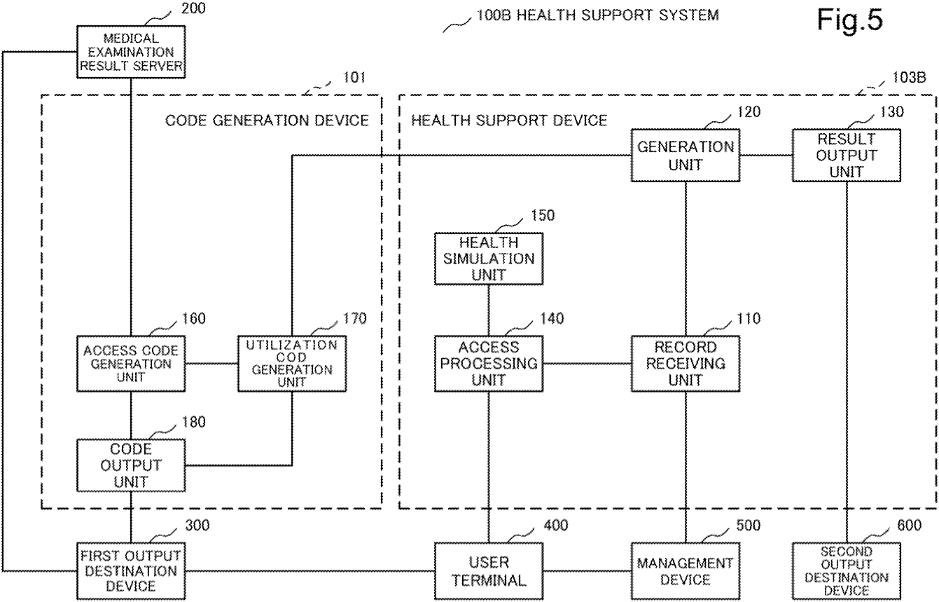

FIG. 5 is a block diagram illustrating a third example of a configuration of the health support system according to the second example embodiment of the present disclosure. In the example of FIG. 5, a health support system 100B includes a code generation device 101 and a health support device 103B. The code generation device 101 is the same as the code generation device 101 illustrated in FIG. 4. The health support device 103B includes the record receiving unit 110, the generation unit 120, the result output unit 130, the access processing unit 140, and health simulation unit 150.

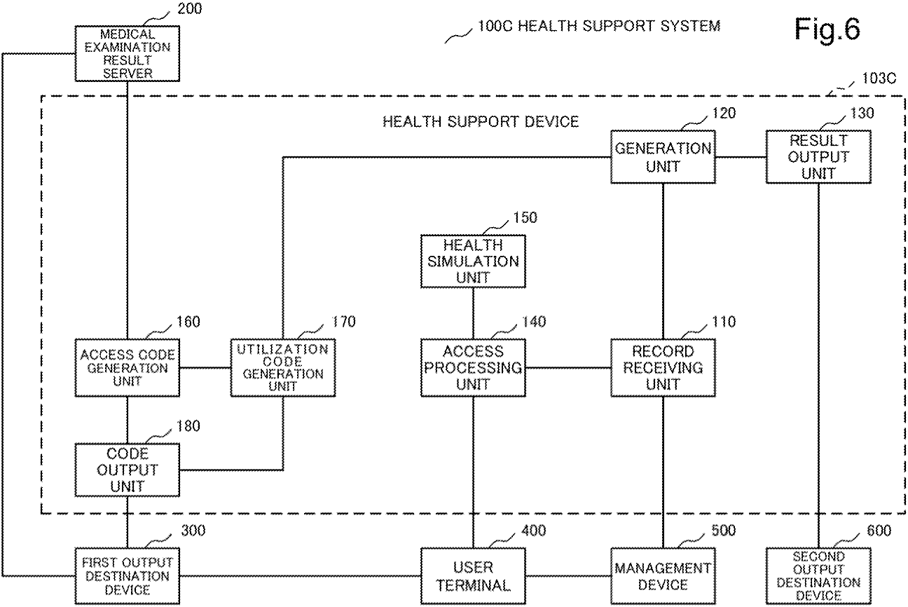

FIG. 6 is a block diagram illustrating a fourth example of the configuration of the health support system according to the second example embodiment of the present disclosure. In the example of FIG. 6, a health support system 100C includes a health support device 103C. The health support device 103 includes a record receiving unit 110, a generation unit 120, a result output unit 130, an access processing unit 140, a health simulation unit 150, an access code generation unit 160, a utilization code generation unit 170, and a code output unit 180.

The configuration of the health support system is not limited to the above example.

### Operation

Next, an operation of the health support system 100 according to the second example embodiment of the present disclosure will be described in detail with reference to the drawings.

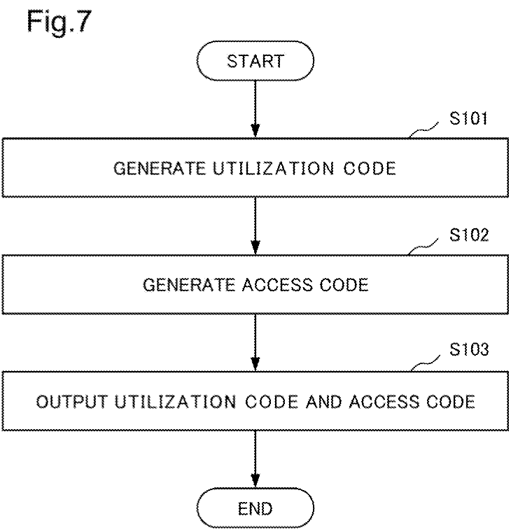

FIG. 7 is a flowchart illustrating an example of an operation of generating a code of the health support system 100 according to the second example embodiment of the present disclosure. Upon receiving the results of the medical examination for each user from the medical examination result server 200, the health support system 100 starts the operation illustrated in FIG. 7.

In the example illustrated in FIG. 7, first, the utilization code generation unit 170 generates the utilization code (step S101). Next, the access code generation unit 160 generates the access code using the results of the medical examination for each user (step S102). The code output unit 180 outputs the utilization code and the access code (step S103). The health support system 100 may first perform the operation of step S102, and may perform the operation of step S101 after the operation of step S102.

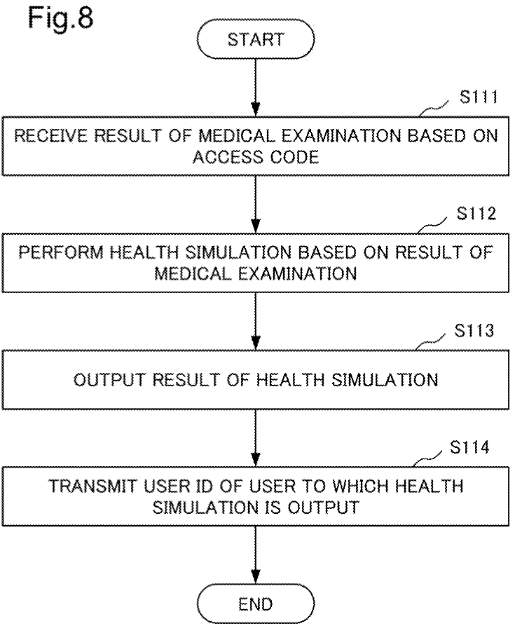

FIG. 8 is a flowchart illustrating an example of an operation of performing the health simulation of the health support system 100 according to the second example embodiment of the present disclosure. In the example illustrated in FIG. 8, the access processing unit 140 receives the result of the medical examination based on the access code from the user terminal 400 (step S111). The result of the medical examination based on the access code represents the result of the medical examination represented by the access code. Next, the health simulation unit 150 performs the health simulation based on the result of the medical examination (step S112). The access processing unit 140 outputs the result of the health simulation to the user terminal 400 (step S113). The access processing unit 140 further transmits the user ID (that is, the user ID of the user on which the health simulation has been executed) of the user from which the health simulation is output to record receiving unit 110 (step S114).

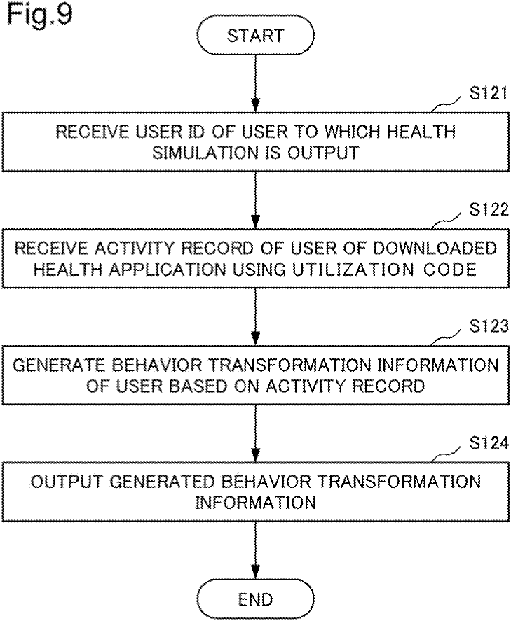

FIG. 9 is a flowchart illustrating an example of an operation of generating the behavior transformation information of the health support system 100 according to the second example embodiment of the present disclosure. In the example illustrated in FIG. 9, the record receiving unit 110 receives the user ID of the user to which the health simulation is output from the access processing unit 140 (step S121). The record receiving unit 110 receives the activity record of the user of the health application downloaded using the utilization code from the management device 500 (step S122). Next, the generation unit 120 generates the behavior transformation information of the user based on the activity record (step S123). The result output unit 130 outputs the generated behavior transformation information (step S124).

### Effect

The present example embodiment has the same effect as that of the first example embodiment. The reason is the same as the reason why the effects of the first example embodiment are generated.

### Specific Example of Behavior Transformation Information

The behavior transformation of the present example embodiment may be at least one of the behavior transformation of the first example embodiment and behavior transformation exemplified below. The behavior transformation information of the present example embodiment may be at least one of the behavior transformation information of the first example embodiment and the behavior transformation information exemplified below.

In the present example embodiment, the behavior transformation may be registration of a service of the health application in cooperation with the execution of the health simulation by the access code. In this case, the activity record may be a record of registration of the health application. The record of the registration of the health application may include the user ID of the user who has registered the service of the health application, the registration date and time, and the information indicating whether the result of the medical examination at the time of registration is provided.

The registration of the service of the health application in cooperation with the execution of the health simulation by the access code is, for example, providing the result of the medical examination to the management device 500 upon registering the service of the health application. In this case, upon registering the service of the health application, the health application transmits, for example, the result of the medical examination generated from the access code to the management device 500, in addition to the user ID. Upon registering the service of the health application to the record receiving unit 110, the management device 500 transmits the user ID of the user who has provided the result of the medical examination to the record receiving unit 110. The record receiving unit 110 receives the user ID of the user who has provided the result of the medical examination upon registering the service of the health application from the management device 500, and transmits the received user ID to the generation unit 120. In this case, the record receiving unit 110 may not receive the user ID from the access processing unit 140. The access processing unit 140 may not receive the user ID from the user terminal 400. The access code may not represent the user ID.

Upon registering the service of the health application, the generation unit 120 receives the user ID of the user who has provided the result of the medical examination from the record receiving unit 110. The generation unit 120 calculates the number of users who has provided the result of the medical examination upon registering the service of the health application. The generation unit 120 may set, as the behavior transformation information, a transition of the number of users who have provided the result of the medical examination upon registering the service of the health application or the number of users who has provided the result of the medical examination upon registering the service of the health application.

The generation unit 120 may calculate a ratio of the number of users who have provided the result of the medical examination upon registering the service of the health application to the number of users whose utilization codes have been generated (that is, the number of user IDs whose utilization codes have been generated). The generation unit 120 may set, as the behavior transformation information, a ratio of the number of users who have provided the result of the medical examination upon registering the service of the health application to the number of users whose utilization codes have been generated, or a transition of the ratio.

Hereinafter, the user ID of the user who has registered the service of the health application in cooperation with the execution of the health simulation by the access code is referred to as a transformant ID. The registration of the service of the health application in association with the execution of the health simulation by the access code may be executing the health simulation by the access code and executing the registration of the service of the health application downloaded using the utilization code.

In this case, the generation unit 120 receives the user ID (hereinafter, referred to as a registrant ID) of the user who has registered the service of the health application from the management device 500 via the record receiving unit 110. The user ID (hereinafter, referred to as an executor ID) included in both the user ID for which the health simulation using the access code was executed and the registrant ID is specified as the transformant ID. The generation unit 120 may calculate the number of users (that is, the number of transformant IDs) indicated by the transformant ID or a transition thereof as the behavior transformation information. The generation unit 120 may calculate, as the behavior transformation information, a ratio of the number of users indicated by the transformant ID to the number of users whose utilization codes have been generated (that is, the number of user IDs for which the utilization codes have been generated) or the transition of the ratio.

The registration of the service of the health application in cooperation with the execution of the health simulation by the access code may be executing the health simulation by the access code and executing the registration of the service of the health application downloaded using the utilization code. In this case, the access processing unit 140 transmits, to the record receiving unit 110, a combination (hereinafter, referred to as execution time information) of the user ID of the user who has performed the request for the health simulation by the access code and the time when the request has been received. Furthermore, the management device 500 transmits, to the record receiving unit 110, a combination (hereinafter, referred to as registration time information) of the user ID of the user who has registered the health application and the time when the registration has been performed. In this case, the record receiving unit 110 receives a combination of the user ID of the user who has performed the health simulation request by the access code and the time when the request has been received. The record receiving unit 110 receives a combination of the user ID of the user who has registered the health application and the time when the registration has been performed. Then, the record receiving unit 110 transmits, as a part of the activity record, the combination of the user ID of the user who has made the request for the health simulation by the access code and the time when the request has been received to the generation unit 120. The record receiving unit 110 transmits, as a part of the activity record, the combination of the user ID of the user who has registered the health application and the time when the registration has been performed to the generation unit 120. The generation unit 120 uses the received execution time information and registration time information to execute the health simulation by the access code, and then extracts the user ID of the user who has registered the service of the health application downloaded using the utilization code as the transformant ID. The generation unit 120 sets the extracted user ID as the transformant ID. The generation unit 120 may calculate the number of users (That is, the number of transformant IDs) indicated by the transformant ID or a transition thereof as the behavior transformation information. The generation unit 120 may calculate, as the behavior transformation information, a ratio of the number of users indicated by the transformant ID to the number of users whose utilization codes have been generated (that is, the number of user IDs for which the utilization codes have been generated) or the transition of the ratio.

### First Modified Example

Next, a first modified example of the second example embodiment of the present disclosure will be described in detail with reference to the drawings.

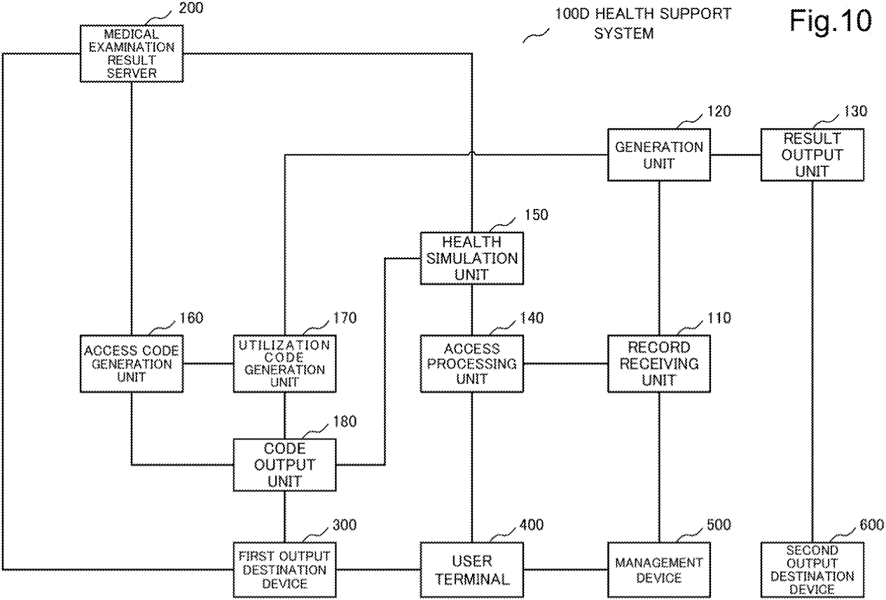

FIG. 10 is a block diagram illustrating an example of a configuration of a health support system according to a first modified example of the second example embodiment of the present disclosure. A health support system 100D of the present modified example is the same as the health support system 100 of the second example embodiment except for differences described below.

In the present modified example, the code output unit 180 requests the health simulation unit 150 to perform the health simulation of the user (that is, user whose access code and utilization code are generated) whose utilization code is generated. Specifically, the code output unit 180 transmits the user ID of the user whose utilization code is generated to the health simulation unit 150.

When the health simulation of the user whose utilization code is generated is requested from the code output unit 180, the health simulation unit 150 downloads the result of the medical examination of the user from the medical examination result server 200. Specifically, the health simulation unit 150 receives the user ID from the code output unit 180, and transmits a request for the result of the medical examination including the received user ID to the medical examination result server 200. The medical examination result server 200 receives the request for the result of the medical examination including the user ID, and transmits the result of the medical examination of the user indicated by the user ID included in the received request to the health simulation unit 150. The health simulation unit 150 receives the result of the medical examination and performs the health simulation using the received result of the medical examination. The health simulation unit 150 transmits the result of the health simulation to the code output unit 180.

The code output unit 180 receives the result of the health simulation, and generates an information providing sheet including the access code, the utilization code, and the result of the health simulation. In the present modified example, the information providing sheet may include the result of the health simulation in addition to the access code, the utilization code, and the result of the medical examination.

The code output unit 180 may receive the result of the user’s medical examination from the access code generation unit 160 and transmit the received result of the medical examination to the health simulation unit 150. In this case, the health simulation unit 150 may not request the result of the medical examination to the medical examination result server 200. The medical examination result server 200 may not receive the request for the result of the medical examination from the health simulation unit 150. The medical examination result server 200 may not transmit the result of the medical examination to the health simulation unit 150. The health simulation unit 150 may not receive the result of the medical examination from the medical examination result server 200.

### Second Modified Example

The utilization code generation unit 170 may generate the user ID. In this case, the utilization code generation unit 170 supplies the generated user ID to the access code generation unit 160 and the generation unit 120. As described above, the access code generation unit 160 supplies the user ID to the medical examination result server 200.

The generation unit 120 may generate the user ID. In this case, for example, when the access code generation unit 160 receives the results of the medical examination for each user, the generation unit 120 may request the generation unit to generate the user ID of the user whose result of the medical examination is included in the received result of the medical examination. When the generation of the user ID is requested, the generation unit 120 may generate the user ID and supply the generated user ID to the access code generation unit 160 and the utilization code generation unit 170. As described above, the access code generation unit 160 supplies the user ID to the medical examination result server 200.

The medical examination result server 200 may assign the user ID to each user. In that case, the medical examination result server 200 supplies the result of the medical examination to which the user ID is assigned instead of the data ID to the access code generation unit 160 for each user. The access code generation unit 160 supplies the user ID to the utilization code generation unit 170 and the generation unit 120. The access code generation unit 160 may directly supply the user ID to the generation unit 120. The access code generation unit 160 may supply the user ID to the generation unit 120 via the utilization code generation unit 170.

### Other Example Embodiments

The health support system according to the example embodiment of the present disclosure can be achieved by a computer including a processor that executes a program loaded in a memory. The health support system according to the example embodiment of the present disclosure can also be achieved by dedicated hardware such as a circuit. The health support system according to the example embodiment of the present disclosure can also be achieved by a combination of the above-described computer and dedicated hardware such as a circuit.

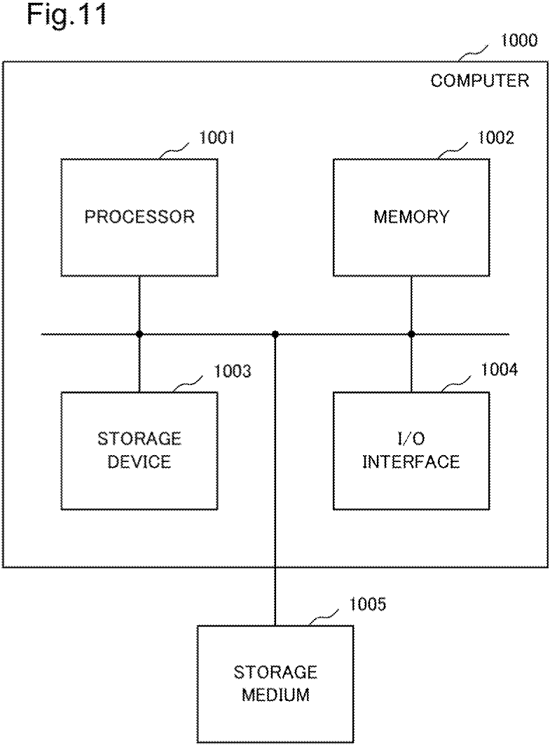

FIG. 11 is a diagram illustrating an example of a hardware configuration of a computer 1000 that can achieve each of the health support systems according to the example embodiment of the present disclosure. In the example illustrated in FIG. 11, the computer 1000 includes a processor 1001, a memory 1002, a storage device 1003, and an input/output (I/O) interface 1004. In addition, the computer 1000 can access a storage medium 1005. The memory 1002 and the storage device 1003 are, for example, storage devices such as a random access memory (RAM) and a hard disk. The storage medium 1005 is, for example, a storage device such as a RAM or a hard disk, a read only memory (ROM), or a portable storage medium. The storage device 1003 may be the storage medium 1005. The processor 1001 can read and write data and programs from and in the memory 1002 and the storage device 1003. The processor 1001 can communicate with, for example, the medical examination result server 200, the first output destination device 300, the user terminal 400, the management device 500, and the second output destination device 600 via the I/O interface 1004. The processor 1001 may access the storage medium 1005. The storage medium 1005 stores a program for operating the computer 1000 as the health support system according to the example embodiment of the present disclosure.

The processor 1001 loads a program, which is stored in the storage medium 1005 and causes the computer 1000 to operate as the health support system according to the example embodiment of the present disclosure, into the memory 1002. When the processor 1001 executes the program loaded in the memory 1002, the computer 1000 operates as the health support system according to the example embodiment of the present disclosure.

The record receiving unit 110, the generation unit 120, the result output unit 130, the access processing unit 140, the health simulation unit 150, the access code generation unit 160, the utilization code generation unit 170, and the code output unit 180 can be achieved by, for example, the processor 1001 that executes a program loaded in the memory 1002. A part or all of the record receiving unit 110, the generation unit 120, the result output unit 130, the access processing unit 140, the health simulation unit 150, the access code generation unit 160, the utilization code generation unit 170, and the code output unit 180 can be achieved by a dedicated circuit that achieves the functions of the respective units.

Japanese Patent Application Laid-open Publication No. 2002-063278 is a technique for selecting an instruction item regarding health. The technique disclosed in Japanese Patent Application Laid-open Publication No. 2010-113668 is a technique for selecting an instruction item related to health. The technique disclosed in Japanese Patent Application Laid-open Publication No. 2014-119817 is a technique for simulating a health index and a health risk. In the techniques of Japanese Patent Application Laid-open Publication No. 2002-063278, Japanese Patent Application Laid-open Publication No. 2010-113668, and Japanese Patent Application Laid-open Publication No. 2014-119817, it is not possible to specify whether the subject actually transforms the behavior.

The present disclosure has the effect of grasping information on a transformation of subject’s behavior related to health.

Some or all of the above embodiments may be described as the following supplementary notes, but are not limited thereto.

### Supplementary Note 1

A health support system, including:
a record receiving unit configured to receive an activity record representing behavior of a user of a health application downloaded using a utilization code for downloading the health application, the utilization code being attached to information on a result of medical examination;
a generation unit configured to detect a transformation of the behavior of the user from the received activity record, and generate behavior transformation information representing the transformation; and
a result output unit configured to output the generated behavior transformation information.

### Supplementary Note 2

The health support system according to supplementary note 1, wherein

the generation unit generates the behavior transformation information from the activity record including information on the user who starts using the health application, the behavior transformation information including statistical information on the number of users who starts using the health application using the utilization code.

### Supplementary Note 3

The health support system according to supplementary note 1 or 2, in which

the generation unit generates the behavior transformation information from the activity record including a record of usage of the health application, the behavior transformation information including a state of continuation of transformed behavior after a transformation of a predetermined-type behavior by the user.

### Supplementary Note 4

The health support system according to any one of supplementary notes 1 to 3, wherein

the generation unit generates the behavior transformation information from a period of an event and the activity record, the behavior transformation information including information on the transformation of the behavior of the user, the transformation being caused by the event.

### Supplementary Note 5

The health support system according to any one of supplementary notes 1 to 4, wherein

the generation unit generates the behavior transformation information from the activity record including a record of achievement of a health mission by the user, the behavior transformation information including a state of achievement of the health mission by the user.

### Supplementary Note 6

The health support system according to any one of supplementary notes 1 to 5, wherein

the generation unit generates the behavior transformation information from the activity record including a record of information provision by the health application and a reaction of the user to the information provision, the behavior transformation information including a state of the reaction by the user to the information provision by the health application.

### Supplementary Note 7

The health support system according to any one of supplementary notes 1 to 6, wherein
the generation unit generates the behavior transformation information for the user individually, and
the result output unit outputs the behavior transformation information of the user to a server that sends the behavior transformation information to a terminal device executing the health application used by the user.

### Supplementary Note 8

The health support system according to any one of supplementary notes 1 to 7, wherein

the generation unit generates the behavior transformation information from the activity record including a period of the event , the behavior transformation information including the information on the transformation of the behavior of the user individually for the user, the transformation being caused by the event.

### Supplementary Note 9

The health support system according to any one of supplementary notes 1 to 8, wherein
the result output unit further outputs an access code for accessing information on the result of the medical examination, and
the generation unit generates statistical information on a count of users who downloads the health application using the utilization code within a predetermined time after accessing the information on the result of the medical examination by using the access code.

### Supplementary Note 10

The health support system according to supplementary note 9, further including

an access code generation unit configured to generate the access code.

### Supplementary Note 11

The health support system described in any one of supplementary notes 1 to 10, further including

a utilization code generation unit configured to generate the utilization code.

### Supplementary Note 12

A health support method, including:
receiving an activity record representing behavior of a user of a health application downloaded using a utilization code for downloading the health application, the utilization code being attached to information on a result of medical examination;
detecting a transformation of the behavior of the user from the received activity record, and generating behavior transformation information representing the transformation; and
outputting the generated behavior transformation information.

### Supplementary Note 13

The health support method according to supplementary note 12, further including

generating the behavior transformation information from the activity record including information on the user who starts using the health application, the behavior transformation information including statistical information on a count of users who starts using the health application using the utilization code.

### Supplementary Note 14

The health support method according to supplementary note 12 or 13, further including

generating the behavior transformation information from the activity record including the record of the use of the health application, the behavior transformation information including a state of continuation of transformed behavior after the transformation of predetermined-type behavior by the user.

### Supplementary Note 15

The health support method according to any one of supplementary notes 12 to 14, further including

generating the behavior transformation information from a period of an event and the activity record, the behavior transformation information including information on the transformation of the behavior of the user, the transformation being caused by the event.

### Supplementary Note 16

The health support method according to any one of supplementary notes 12 to 15, further including

generating the behavior transformation information from the activity record including a record of achievement of the health mission by the user, the behavior transformation information including a state of achievement of a health mission by the user.

### Supplementary Note 17

The health support method according to any one of supplementary notes 12 to 16, further including

generating the behavior transformation information from the activity record including a record of information provision by the health application and the reaction of the user to the information provision, the behavior transformation information including a state of the reaction by the user to the information provision by the health application.

### Supplementary Note 18

The health support method according to any one of supplementary notes 12 to 17, further including:
generating the behavior transformation information for the user independently; and
outputting the behavior transformation information of the user to a server that sends the behavior transformation information to a terminal device executing the health application used by the user.

### Supplementary Note 19

The health support method according to any one of supplementary notes 12 to 18, further including

generating the behavior transformation information from the activity record including a period of the event, the behavior transformation information including the information on the transformation of the behavior independently for the user, the transformation being caused by the event.

### Supplementary Note 20

The health support method according to any one of supplementary notes 12 to 19, further including
outputting an access code for accessing the information on the result of the medical examination; and
generating statistical information on a count of users who download the health application using the utilization code within a predetermined time after accessing the information on the result of the medical examination by using the access code.

### Supplementary Note 21

The health support method according to supplementary note 20, further including generating the access code.

### Supplementary Note 22

The health support method according to any one of supplementary notes 12 to 21, further including generating the utilization code.

### Supplementary Note 23

A program causing a computer to execute:
record receiving processing of receiving an activity record representing behavior of a user of a health application downloaded using a utilization code for downloading the health application, the utilization code being attached to information on a result of medical examination;
generation processing of detecting a transformation of the behavior of the user from the received activity record, and generating behavior transformation information representing the transformation; and
result output processing of outputting the generated behavior transformation information.

### Supplementary Note 24

The program according to supplementary note 23, in which

the generation processing generates the behavior transformation information from the activity record including information on the user who starts using the health application, the behavior transformation information including statistical information on a count of users who starts using the health application using the utilization code.

### Supplementary Note 25

The program according to supplementary note 23 or 24, wherein

the generation processing generates the behavior transformation information from the activity record including a record of usage of the health application, the behavior transformation information including a state of continuation of transformed behavior after the transformation of predetermined-type behavior by the user.

### Supplementary Note 26

The program according to any one of supplementary notes 23 to 25, wherein

the generation processing generates the behavior transformation information from a period of an event and the activity record, the behavior transformation information including information on the transformation of the behavior of the user, the transformation being caused by the event.

### Supplementary Note 27

The program according to any one of supplementary notes 23 to 26, wherein

the generation processing generates the behavior transformation information from the activity record including a record of achievement of a health mission by the user, the behavior transformation information including a state of achievement of the health mission by the user.

### Supplementary Note 28

The program according to any one of supplementary notes 23 to 27, wherein

the generation processing generates the behavior transformation information from the activity record including a record of information provision by the health application and a reaction of the user to the information provision, the behavior transformation information including a state of the reaction by the user to the information provision by the health application.

### Supplementary Note 29

The program according to any one of supplementary notes 23 to 28, wherein
the generation processing generates the behavior transformation information for the user individually, and
the result output processing outputs the behavior transformation information of the user to a server that sends the behavior transformation information to a terminal device executing the health application used by the user.

### Supplementary Note 30

The program according to any one of supplementary notes 23 to 29, wherein

the generation processing generates the behavior transformation information from the activity record including a period of an event, the behavior transformation information including the information on the transformation of the behavior of the user individually, the transformation being caused by the event.

### Supplementary Note 31

The program according to any one of supplementary notes 23 to 30, wherein
the result output processing further outputs an access code for accessing information on the result of the medical examination, and
the generation process generates statistical information on a count of users who downloads the health application by using the utilization code within a predetermined time after accessing the information on the result of the medical examination by using the access code.

### Supplementary Note 32

The program according to supplementary note 31, the program causing the computer to execute access code generation processing of generating the access code.

### Supplementary Note 33

The program according to any one of supplementary notes 23 to 32, the program causing the computer to execute utilization code generation processing of generating the utilization code.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims.

1. A health support system comprising:
at least one memory configured to store instructions; and
at least one processor configured to execute the instructions to:
receive an activity record representing behavior of a user of a health application downloaded using a utilization code for downloading the health application, the utilization code being attached to information on a result of medical examination;
detect a transformation of the behavior of the user from the received activity record;
generate behavior transformation information representing the transformation; and
output the generated behavior transformation information.

2. The health support system according to claim 1, wherein
the at least one processor is further configured to execute the instructions to
generate the behavior transformation information from the activity record including information on the user who starts using the health application, the behavior transformation information including statistical information on a count of users who starts using the health application using the use code.

3. The health support system according to claim 1, wherein
the at least one processor is further configured to execute the instructions to
generate the behavior transformation information from the activity record including a record of usage of the health application, the behavior transformation information including a state of continuation of transformed behavior after the transformation of predetermined-type behavior by the user.

4. The health support system according to claim 1, wherein
the at least one processor is further configured to execute the instructions to
generate the behavior transformation from a period of an event and the activity record, the behavior transformation information including information on the transformation of the behavior of the user, the transformation being caused by the event.

5. The health support system according to claim 1, wherein
the at least one processor is further configured to execute the instructions to
generate the behavior transformation information from the activity record including a record of achievement of a health mission by the user, the behavior transformation information including a state of achievement of the health mission by the user.

6. The health support system according to claim 1, wherein
the at least one processor is further configured to execute the instructions to
generate the behavior transformation information from the activity record including a record of information provision by the health application and a reaction of the user to the information provision, the behavior transformation information including a state of the reaction by the user to the information provision by the health application.

7. The health support system according to claim 1, wherein
the at least one processor is further configured to execute the instructions to:
generate the behavior transformation information for the user individually: and
output the behavior transformation information of the user to a server that sends the behavior transformation information to a terminal device executing the health application used by the user.

8. The health support system according to claim 1 wherein
the at least one processor is further configured to execute the instructions to:
further output an access code for accessing information on the result of the medical examination; and
generate statistical information on a count of users who download the health application using the utilization code within a predetermined time after accessing the information on the result of the medical examination by using the access code.

9. A health support method comprising:
receiving an activity record representing behavior of a user of a health application downloaded using a utilization code for downloading the health application, the utilization code being attached to information on a result of medical examination;
detecting a transformation of the behavior of the user from the received activity record;
generating behavior transformation information representing the transformation; and
outputting the generated behavior transformation information.

10. The health support method according to claim 9, wherein
generating the behavior transformation information includes generating the behavior transformation information from the activity record including information on the user who starts using the health application, the behavior transformation information including statistical information on a count of users who starts using the health application using the use code.

11. The health support method according to claim 9, wherein
generating the behavior transformation information includes generating the behavior transformation information from the activity record including a record of usage of the health application, the behavior transformation information including a state of continuation of transformed behavior after the transformation of predetermined-type behavior by the user.

12. The health support method according to claim 9, wherein
generating the behavior transformation information includes generating the behavior transformation from a period of an event and the activity record, the behavior transformation information including information on the transformation of the behavior of the user, the transformation being caused by the event.

13. The health support method according to claim 9, wherein
generating the behavior transformation information includes generating the behavior transformation information from the activity record including a record of achievement of a health mission by the user, the behavior transformation information including a state of achievement of the health mission by the user.

14. The health support method according to claim 9, wherein
generating the behavior transformation information includes generating the behavior transformation information from the activity record including a record of information provision by the health application and a reaction of the user to the information provision, the behavior transformation information including a state of the reaction by the user to the information provision by the health application.

15. The health support method according to claim 9, wherein
generating the behavior transformation information includes generating the behavior transformation information for the user individually, and
outputting the behavior transformation information includes outputting the behavior transformation information of the user to a server that sends the behavior transformation information to a terminal device executing the health application used by the user.

16. The health support method according to claim 9, the health support method further comprising:
further outputting an access code for accessing information on the result of the medical examination; and
generating statistical information on a count of users who download the health application using the utilization code within a predetermined time after accessing the information on the result of the medical examination by using the access code.

17. A non-transitory computer-readable storage medium storing a program causing a computer to execute:
record receiving processing of receiving an activity record representing behavior of a user of a health application downloaded using a utilization code for downloading the health application, the utilization code being attached to information on a result of medical examination;
generation processing of detecting a transformation of the behavior of the user from the received activity record, and generating behavior transformation information representing the transformation; and
record output processing of outputting the generated behavior transformation information.

18. The non-transitory computer-readable storage medium according to claim 17, wherein
the generation processing generates the behavior transformation information from the activity record including information on the user who starts using the health application, the behavior transformation information including statistical information on a count of users who starts using the health application using the use code.

19. The non-transitory computer-readable storage medium according to claim 17, wherein
the generation processing generates the behavior transformation information from the activity record including a record of usage of the health application, the behavior transformation information including a state of continuation of transformed behavior after the transformation of predetermined-type behavior by the user.

20. The non-transitory computer-readable storage medium according to claim 17, wherein
the generation processing generates the behavior transformation from a period of an event and the activity record, the behavior transformation information including information on the transformation of the behavior of the user, the transformation being caused by the event.
